(19) United States
(12) Patent Application Publication
(10) Pub. No.: US 2010/0168087 A1
(43) Pub. Date: Jul. 1, 2010

(54) BENZO[1,2,3]THIADIAZINE DERIVATES

(76) Inventors: Marta Porcs-Makkay, Pomaz (HU); Gyula Lukacs, Budapest (HU); Gabor Kapus, Pecel (HU); Istvan Gacsalyi, Budapest (HU); Gyula Simig, Budapest (HU); Gyoergy Levay, Budakeszi (HU); Tibor Mezei, Budapest (HU); Miklos Vegh, Budapest (HU); Szabolcs Kertesz, Fot (HU); Jozsef Barkoczy, Budapest (HU); Csilla Leveleki, Budapest (HU); Laszlo Gabor Harsing, Budapest (HU)

Correspondence Address: KF ROSS PC, 5683 RIVERDALE AVENUE, SUITE 203 BOX 900, BRONX, NY, 10471-0900, US

(21) Appl. No.: 12/377,771

(22) PCT Filed: Aug. 13, 2007
(86) PCT No.: PCT/HU07/00071

§ 371 Date: Jul. 29, 2009

(30) Foreign Application Priority Data
Aug. 16, 2006 (HU) P0600650
Aug. 2, 2007 (HU) P0700504

(51) Int. Cl. A61K 31/5415; C07D 285/16; A61P 25/00

(52) U.S. Cl. 514/222.8; 544/11

(57) ABSTRACT

The present invention relates to new benzo[1,2,3]thiadiazine-1,1-dioxide derivatives of the general Formula (I), medicaments containing such compounds and the use thereof in the medicine. Benzo[1,2,3]thiadiazine-1,1-dioxide derivatives of the general Formula (I) are suitable for the prevention or treatment of the diseases of the central nervous system.

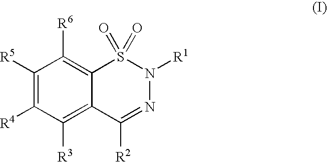

### FIELD OF THE INVENTION

The present invention relates to new benzo[1,2,3]thiadiazine-1,1-dioxide derivatives of the general Formula (I), medicaments containing said derivatives, process for the preparation thereof and the use of the new compounds in the medicine.

More specifically, the present invention relates to benzo[1,2,3]thiadiazine-1,1-dioxide derivatives of the general Formula (I),

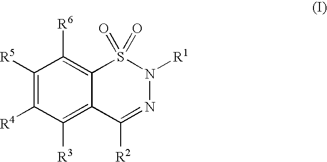

wherein
R¹ and R² represent independently hydrogen, a straight- or branched-chain alkyl group having 1 to 4 carbon atoms;
R³, R⁴, R⁵ and R⁶ represent independently hydrogen, halogen, a straight- or branched-chain alkyl group having 1 to 4 carbon atoms or an alkoxy group containing a straight- or branched-chain alkyl group having 1 to 4 carbon atoms.

### TECHNICAL BACKGROUND OF THE INVENTION

The benzo[1,2,3]thiadiazine-1,1-dioxide ring system has been prepared for the first time in 1917 (E. Schrader, J. Prakt. Chem. 1918, 96, 180-185.), when the ring closure was performed by reacting 2-ciano-benzenesulfonylchloride with hydrazine yielding 4-hydrazino-substituted benzo[1,2,3]thiadiazine-1,1-dioxide (Reaction Scheme 1).

A similar process is known from the state of the art for the preparation of diuretic 4-hydrazino-benzo[1,2,3]thiadiazine-1,1-dioxide derivatives (P. Schmidt, K. Eichenberger, M. Wilhelm, Helv. Chim. Acta 1962, 45, 996-999.).

The unsubstituted benzo[1,2,3]thiadiazine-1,1-dioxide structural unit can be prepared by two methods starting from sodium o-formyl-benzenesulfonate (J. F. King, A. Hawson, D. M. Deaken, J. Komery, J. C. S. Chem. Comm. 1969, 33-34; J. F. King, A. H. Huston, J. Komery, D. M. Deaken, D. R. K. Harding, Can. J. Chem 1971, 49, 936-942). The two reaction routes are demonstrated in Reaction Scheme 2.

The first process comprises reacting sodium o-formyl-benzenesulfonate with thionylchloride and the thus obtained o-formyl-benzenesulfonylchloride is reacted with hydrazine, resulting in the formation of benzo[1,2,3]thiadiazine-1,1-dioxide.

According to the second process, the sodium salt of o-formyl-benzenesulfonic acid is reacted with hydrazine and the thus obtained hydrazide is subjected to ring closure in the presence of phosphorous pentachloride or phosphorous oxychloride and hydrazine, yielding benzo [1,2,3]thiadiazine-1,1-dioxide.

4-phenyl-benzo[1,2,3]thiadiazine-1,1-dioxide derivatives are prepared according to the state of the art starting from o-amino-benzophenone. β-amino-benzophenone is converted into a diazonium salt, which is subsequently transformed into the corresponding sulfochloride by reacting the diazonium salt with sulfur dioxide in the presence of copper(I)-salts. The sulfochloride is subsequently converted into 4-phenyl-benzo[1,2,3]thiadiazine-1,1-dioxide using one of the two methods described above (J. B. Wright, J. Het. Chem. 1968, 5, 453-459).

Certain 4-phenyl substituted benzo[1,2,3]thiadiazine-1,1-dioxide derivatives were disclosed in U.S. Pat. No. 3,407,197. The compounds disclosed in said patent showed disinfectant, pesticide and herbicide effect.

In U.S. Pat. No. 3,407,198,2-alkylated benzo[1,2,3]thiadiazine-1,1-dioxide derivatives are disclosed, which are claimed to exert effects in the central nervous system. However, the patent is silent about the specific disclosure regarding said effects. The reaction scheme of the preparation of the compounds is demonstrated in Reaction Scheme 3.

According to the state of the art, there have been disclosed 4-phenylbenzo[1,2,3]thiadiazine-1,1-dioxide derivatives exhibiting diuretic effect (O. B. T. Nielsen, C. K. Nielsen, P. W. Feit, J. Med. Chem. 1973, 16, (10); 1170-1177.). The preparation of said compounds is carried out essentially by following the Wright-process described above. The reaction route is demonstrated in Reaction Scheme 4.

Published International Patent Application WO 99/67240 discloses antibiotic carbapenem derivatives substituted with benzo[1,2,3]thiadiazine-1,1-dioxide derivatives in position 2.

Due to the significantly increased rate of economical-technological development and the corresponding social changes, the civilized man is subject to many sources of stress. As a result of exposure to different stress sources, a disease or disorder belonging to the group of anxiety disorders can more and more often develop.

Anxiety is a characteristic symptom of central nervous system origin which can be resulted from psychiatric disease, medicinal condition, trauma, or surgical intervention as well. Anxiety is most often treated by the administration of medicines belonging to the benzodiazepine group, e.g. diazepam, chlorodiazepoxide, alprazolam.

Although there are several pharmaceutically active ingredients available for the treatment of the group of anxiety disorders, such active ingredients exhibit several non-desired side effects regarding the life quality of the patient. Furthermore, it is known fact that the most beneficial therapeutic method in case of several diseases of the central nervous system is the combination therapy. Thus, there exists a continuous need for the development of new pharmaceutically active ingredients showing anxiolytic activity.

The objective of our research-development work was to develop new pharmaceutically active ingredients suitable for the use in medicaments for the treatment or prevention of the diseases, disorders or conditions belonging to the group of anxiety disorders including generalized anxiety disorder, panic disorder, agoraphobia, social phobia, other types of phobias, posttraumatic stress disorder and those diseases of the central nervous system which are accompanied by the symptoms of anxiety.

The above objective is solved by the present invention.

### DETAILED DESCRIPTION OF THE INVENTION

The basis of the present invention resides in the surprising recognition that the compounds of the general Formula (I), wherein
R¹ and R² represent independently hydrogen, a straight- or branched-chain alkyl group having 1 to 4 carbon atoms,
R³, R⁴, R⁵ and R⁶ each represents hydrogen, halogen, a straight- or branched-chain alkyl group having 1 to 4 carbon atoms or an alkoxy group containing a straight- or branched-chain alkyl group having 1 to 4 carbon atoms,
   show significant anxiolytic effect and therefore said compounds are suitable for the treatment or prevention of the diseases or conditions belonging to the group of anxiety disorders including generalized anxiety disorder, panic disorder, agoraphobia, social phobia, other types of phobias, posttraumatic stress disorder. Furthermore, the compounds of the general Formula (I) are useful in the treatment or prevention of those diseases of the central nervous system which are associated with or manifested in the symptoms of anxiety, including attention-deficit hyperactivity, anorexia, bulimia, compulsive disorders, obsessive-compulsive disorder and disturbances of the sexual function. The compounds of the general Formula (I) are new.

The anxiolytic effect of the benzo[1,2,3]thiadiazine-1,1-dioxide derivatives of the general Formula (I) is especially surprising since it can not be brought into relation with the diuretic, insecticide or herbicide effect of the analogous 4-phenylbenzo [1,2,3]thiadiazine-1,1-dioxide derivatives known from the prior art. Although it has been disclosed in the prior art that certain representatives of the 4-phenylbenzo[1,2,3]thiadiazine-1,1-dioxides possess activity in the central nervous system, the prior art is silent about specific data regarding the characterization of the type and strength of said activity.

According to the first aspect of the present invention, there are provided new benzo[1,2,3]thiadiazine-1,1-dioxide derivatives of the Formula (I), wherein

R¹ and R² each represents hydrogen, a straight- or branched-chain alkyl group having 1 to 4 carbon atoms;
R³, R⁴, R⁵ and R⁶ represent independently hydrogen, halogen, straight- or branched-chain alkyl or an alkoxy containing a straight- or branched-chain alkyl having 1 to 4 carbon atoms.

In the present description, the expression “halogen” means fluorine, bromine, iodine or chlorine.

The expression “a straight- or branched-chain alkyl group comprising 1 to 4 carbon atoms” is understood as a saturated hydrocarbon group having 1 to 4 carbon atoms, e.g. methyl, ethyl, n-propyl, isopropyl, n-butyl, sec-butyl, tert-butyl, isobutyl.

The meaning of the expression “an alkoxy group comprising 1 to 4 carbon atoms” is an alkoxy group which contains a straight- or branched-chain alkyl group of 1 to 4 carbon atoms corresponding to the above definition.

According to a further aspect of the present invention, there is provided a method for the preparation of the compounds of the general Formula (I).

Those representatives of the compounds of the general Formula (I), wherein R¹ represents hydrogen and the meaning of R², R³, R⁴, R⁵, R⁶ is as defined above, can be prepared according to the methods known from the prior art.

The compounds of the general Formula (I), wherein R¹ is hydrogen and the meaning of R², R³, R⁴, R⁵, R⁶ is as defined above, can be prepared by starting from the compounds of the general Formula (II) (Gy. Lukács, M. Porcs-Makkay, Gy. Simig, Tetrahedron Letters 2003, 44, 3211-3214.),

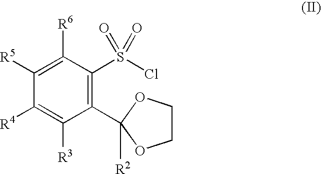

wherein the meaning of R², R³, R⁴, R⁵, R⁶ is as defined above, by reacting said compound with acetyl hydrazine and subjecting the thus obtained product to ring closure under acidic conditions or according to the analogy of the methods known from the prior art (F. Huet, A. Lechevallier, M. Pellet, J. M. Conia, Synthesis 1978, 63-65.) by the ring closure of orto-acylsulfonic acid derivatives of the Formula (III)

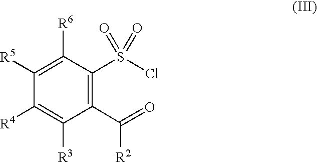

in presence of hydrazine (J. B. Wright, J. Het. Chem. 1968, 5, 453-459.).

Those compounds of the general Formula (I), wherein the meaning of R¹ and R⁶ each is hydrogen, R², R³, R⁴, R⁵ represent the substituents as defined above, can be prepared by reacting a compound of the general Formula (I) wherein R¹ represents hydrogen, R⁶ represents halogen and the meaning of R², R³, R⁴, R⁵ is as defined above, with 1 to 5 molar equivalent amount of an alkyl lithium, preferably with butyl lithium at room temperature or at lower temperature than room temperature, preferably at a temperature between (−78° C.)-(+25° C.) in an inert organic solvent, preferably in diethylether or tetrahydrofurane and thereafter transforming the thus obtained metal-organic product by reacting said product with water into the compound of the general Formula (I).

Those compounds of the general Formula (I), wherein R¹ represents alkyl, the meaning of R², R³, R⁴, R⁵, R⁶ is as defined above, can be prepared by reacting a compound of the general Formula (I) wherein R¹ represents hydrogen, the meaning of R², R³, R⁴, R⁵, R⁶ is as above, analogously to the methods known from the prior art (Houben-Weyl: Amine, XI/1, p. 98; J. B. Wright, J. Het. Chem. 1968, 5, 453-459); with an alkyl halogenide derivative in an organic solvent in the presence of an acid-binding reagent.

Suitable acid-binding reagents are inorganic or organic bases, preferably potassium-tent-butylate or sodium hydride. The reaction can be carried out in polar aprotic solvents, preferably in N,N-dimethylformamide or in tetrahydrofurane.

Pharmacological activity of the compounds of the general Formula (I) has been evaluated in elevated maze test in the rat.

The tests were carried out according to the method of S. Pellow and coworkers (J. Neurosci. Methods. 1985, 14, 149-167). During the test, a wooden-floor cross elevated by 50 cm from the floor was used. The length and the width of the arms of the cross were 100 and 15 cm, respectively. Two opposite arms of the cross are fitted at the ends and both sides with black plexyglass wall of 40 cm height (closed arms).

The central section of the cross measuring 15 by 15 cm is open. The other two opposite arms of the cross are not provided with walls (open arms).

The tests were carried out in male Sprague-Dawley rats weighing 200 to 220 g. After the 60-min pretreatment time (per os treatment), the animal was placed in the center of the maze. During the 5-min measurement time, four variables were recorded: the time spent in the open arms; the time spent in the closed arms; the number of entries into the open arms; the number of entries into the closed arms.

Animals normally avoid the open and light parts of the maze, thereby spending little time therein only. The anxiolytic effect of the compounds of the general Formula (I) is indicated by the increase in the time spent (in seconds) in the open arms and/or by the increase in the number of the entries into the open compartments of the maze. The minimum effective dose (MED) was determined for each tested compounds regarding the time spent in the open arms and the number of entries into the open arms. During the test, the known anxiolytic compound 1-methyl-5-phenyl-7-chloro-1,3-dihydro-2H-1,4-benzodiazepin-2-one (INN: diazepam) of the Formula (IV)

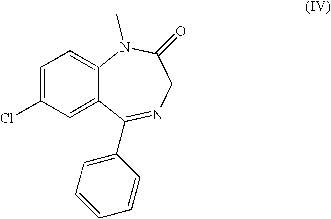

was used. Treatment of the animals was carried out orally with the doses 0.1, 0.3, 1.0, 3.0 for diazepam and 0.01, 0.1, 1.0 mg/kg for the compounds of the general Formula (I).

TABLE 1
Anxiolytic effect of the compounds of the general
Formula (I) in elevated maze test in the rat

| | MED |
|---|---|
| Example No. | (mg/kg p.o.) |
| Diazepam | 1.00 |
| 3 | ≦0.01 |
| 5 | ≦0.01 |
| 17 | ≦0.01 |
| 20 | ≦0.01 |

The test compounds of the general Formula (I) increased the time spent in the open arms and the number of entries into the open arms in the elevated maze test (Table 1). These indicate that the compounds of the general Formula (I) possess significant anxiolytic activity.

On the basis of the above experiments, it can be concluded that the compounds of the general Formula (I) possess entirely unexpected and surprisingly significant anxiolytic effect in a rodent behavioural model. Thus the compounds of the general Formula (I) are suitable for the treatment or prevention of diseases, disorders or states belonging to the group of anxiolytic disorders, i.e. generalized anxiety disorder, panic disorder, agoraphobia, social phobia, other types of phobias, posttraumatic stress disorder and any other disorders of the central nervous system, which are accompanied by or manifested in the symptoms of anxiety. Such disorders include attention-deficit hyperactivity disorder, stress-related adaptation disorder, posttraumatic stress disorder, insomnia, parasomnia, compulsive disorders including obsessive-compulsive disorder, disturbance, of the sexual function, anorexia, bulimia, symptoms of the abuse or withdrawal of drugs or chemical agents, including but not limited to the abuse or withdrawal of alcohol, caffeine, nicotine, sedatives, narcotics, doping agents or drugs of abuse.

The above-mentioned anxiolytic effect is entirely unexpected since the compounds chemically similar to those of the general Formula (I) were known as diuretic, herbicide or pesticide compounds. The anxiolytic effect is not related to the activity of the analogous compounds known from the prior art in any way.

According to a further aspect of the present invention, there are provided medicaments which contain one or more compound(s) of the general Formula (I) and one or more known vehicle(s) or auxiliary agent(s).

The proportion of the active ingredient of the general Formula (I) in the medicaments according to the present invention is generally between 0.1 and 95% by weight, preferably between 5 and 75% by weight.

The medicaments according to the present invention can be administered orally (e.g. powders, tablets, coated tablets, capsules, microcapsules, granules, pellets, dragee, solutions, suspensions or emulsions), parenterally (e.g. in the form of intravenous, intramuscular, subcutaneous or intraperitoneal injections or as infusions), rectally (e.g. as suppositories), transdermally (e.g. as patches), in the form of implants or locally (e.g. creams, ointments, patches). The medicaments according to the present invention can be prepared by the methods of pharmaceutical technology known from the prior art.

Medicaments containing the compounds of the general Formula (I) as active ingredients suitable for oral administration can also contain filling agents or vehicles (e.g. lactose, glucose, starch, calcium phosphate, microcrystalline cellulose), binders (gelatine, sorbitol, polyvinylpyrrollidone), disintegrants (e.g. croscarmellose, sodium carboxymethylcellulose, crospovidone), tabletting aids (e.g. magnesium stearate, talc, polyethyleneglycol, silicic acid, silicon dioxide) or surfactants (e.g. sodium lauryl sulfate).

Liquid medicaments suitable for oral administration containing a compound of the general Formula (I) can be prepared in the form of solutions, suspensions or emulsions and can contain suspending agents (e.g. gelatin; carboxymethylcellulose), emulsifying agents (e.g. sorbitane monooleate), solvents (e.g. water, oils, glycerol, propylene glycol, ethanol), buffers (e.g. acetate, phosphate, citrate buffer) or stabilizing agents (e.g. methyl-4-hydroxybenzoate).

Medicaments suitable for parenteral administration containing a compounds of the general Formula (I) are usually sterile isotonic solution, which can contain pH-adjusting agents and conservants besides the solvent.

The semisolid medicaments containing a compound of the general Formula (I), e.g. suppositories contain the pharmaceutically active ingredient homogeneously dispersed in the suppository base (.e.g. polyethylene glycol, cocoa butter).

The medicaments according to the present invention containing one or more compound(s) of the general Formula (I) as active ingredient(s) can be prepared in the form of modified-, extended-, or controlled-release preparation. Thus the release of the active compound of the general Formula (I) can be provided according to predetermined time function and thus a long-lasting therapeutical effect can be obtained or the frequency of the administration can be decreased. Such modified-, extended- or controlled-release medicaments can be prepared according to methods known from the prior art.

According to a further aspect of the present invention, there is provided a process for the preparation of medicaments containing one or more compound(s) of the general Formula (I) which comprises admixing one or more compound(s) of the general Formula (I) with a pharmaceutically acceptable carrier and if desired, with further auxiliary agents and transforming the thus obtained mixture into a pharmaceutical dosage form. The vehicles and auxiliary agents used in the pharmaceutical technology are known from the prior art (Remington's Pharmaceutical Sciences, Edition 18, Mack Publishing Co., Easton, USA, 1990).

Medicaments containing a compound of the general Formula (I) as active ingredient generally contain the active ingredient in the form of dosage units.

A further aspect of the present invention is the use of benzo[1,2,3]thiadiazine-1,1-dioxide derivatives of the Formula (I) for the treatment or prevention of anxiolytic disorders, i.e. generalized anxiety disorder, panic disorder, agoraphobia, social phobia, other types of phobias, posttraumatic stress disorder and any other disorders of the central nervous system, which are accompanied by the symptoms of anxiety including attention-deficit hyperactivity disorder, stress-related adaptation disorder, posttraumatic stress disorder, insomnia, parasomnia, compulsive disorders including obsessive-compulsive disorders, disturbances of the sexual function, anorexia, bulimia and symptoms of the withdrawal of drugs or chemical agents, including but not limited to the withdrawal of alcohol, caffeine, nicotine, sedatives, narcotics, doping agents or drugs of abuse.

According to another aspect of the present invention, there is provided a method for the treatment or prevention of diseases, disorders or states belonging to the group of anxiolytic disorders, i.e. generalized anxiety disorder, panic disorder, agoraphobia, social phobia, other types of phobias, posttraumatic stress disorder and any other disorders of the central nervous system, which are accompanied by the symptoms of anxiety. Such disorders include attention-deficit hyperactivity disorder, stress-related adaptation disorder, posttraumatic stress disorder, insomnia, parasomnia, compulsive disorders including obsessive-compulsive disorders, disturbances of the sexual function, anorexia, bulimia and symptoms of the withdrawal of drugs or chemical agents, including but not limited to the withdrawal of alcohol, caffeine, nicotine, sedatives, narcotics, doping agents or drugs of abuse, which comprises administering to a patient in need of such treatment a therapeutically effective amount of a benzo[1,2,3]thiadiazine-1,1-dioxide derivative according to the present invention.

The applicable dose depends on several factors including the type and severity of the disease to be treated, the age, physiological status, body weight of the patient and other forms of therapy simultaneously used. The applicable dose should be determined by a physician.

The invention is further demonstrated in the following examples without limiting the invention to said examples.

### Example 1

### 7-chloro-4-methylbenzo[1,2,3]thiadiazine-1,1-dioxide

2-acetyl-5-chlorobenzenesulfonylchloride (5.70 g; 0.0225 mol) is dissolved in tetrahydrofurane (50 ml) and hydrazine monohydrate (8.0 ml; 8.25 g; 0.165 mol) is added dropwise to the solution. The reaction mixture is being heated at its boiling point for one hour. Thereafter the mixture is poured into water and the crystals formed are filtered.

Yield, 2.70 g, white crystals (52%).

Melting point, 187-189° C. (2-propanol)

IR (KBr): 3273 (NH); 1311, 1171 (S=0) cm⁻¹.

¹HNMR (CDCl₃, 400 MHz): 8.26 (1H, s); 7.97 (1H, d, J=2.1 Hz); 7.73 (1H, dd, J=2.2; 8.5 Hz); 7.62 (1H, d, J=8.5 Hz); 2.52 (3H, s) ppm.

¹³CNMR (CDCl₃, 400 MHz): 149.3; 138.2; 135.4; 133.0; 128.0; 126.6; 121.2; 76.7; 20.0 ppm.

Elemental analysis [calculated on the basis of the Formula C₈H₇ClN₂O₂S (230.67)]:

Calculated: C 41.66%; H 3.06%; Cl 15.37%; N 12.14%; S 13.90%

Measured: C 41.72%; H 2.93%; Cl 15.23%; N 12.19%; S 14.10%.

### Example 2

### 4-Ethyl-7-chlorobenzo[1,2,3]thiadiazine-1,1-dioxide

The title compound is prepared according to the procedure of Example 1 starting from 5-chloro-2-propionylbenzenesulfonylchloride (13.9 g; 0.052 mol) and hydrazine monohydrate (4.9 ml; 5.0 g; 0.1 mol).

Yield, 11.7 g, white crystals (92%)

Melting point, 196-198° C. (2-propanol)

IR (KBr): 3132 (NH); 1328, 1181 (S═O) cm⁻¹.

¹HNMR (DMSO, 400 MHz): 12.3 (1H, s); 8.06 (1H, dd, J=1.0; 1.6 Hz); 7.97 (1H, dd, J=9.4; 1.0 Hz); 7.95 (1H, dd, J=1.68.5 Hz); 2.95 (2H, q, J=7.3 Hz); 1.20 (3H, t, J=7.4 Hz) ppm.

¹³CNMR (DMSO, 200 MHz): 152.1; 136.7; 135.1; 133.1; 129.0; 125.8; 120.1; 25.8; 11.4 ppm.

Elemental analysis [calculated on the basis of the Formula C₉H₉ClN₂O₂S (244.70)]:

Calculated: C 44.18%; H 3.71%; Cl 14.49%; N 11.45%; S 13.10%

Measured: C 44.06%; H 3.69%; Cl 14.45%; N 11.47%; S 13.02%.

### Example 3

### 7,8-Dichloro-4-methylbenzo[1,2,3]tiadiazine-1,1-dioxide

The title compound is produced according to the procedure of Example 1 with the difference that 6-acetyl-2,3-dichlorobenzenesulfonylchloride (5.4 g; 0.019 mol) and hydrazine monohydrate (1.9 ml; 2.0 g; 0.04 mol) are used.

Yield, 4.5 g, white crystals (90%)

Melting point, 217-219° C. (ethanol)

IR (KBr): 3155 (NH); 1337, 1162 (S═O) cm⁻¹.

¹HNMR (DMSO, 400 MHz): 12.42 (1H, s); 8.15 (1H, d, J=8.6 Hz); 7.88 (1H, d, J=8.6 Hz); 2.48 (3H, s) ppm.

¹³CNMR (DMSO, 400 MHz): 148.6; 136.3; 134.1; 133.5; 128.5; 128.0; 125.3; 94.1; 20.3 ppm.

Elemental analysis [calculated on the basis of the Formula C₈H₆Cl₂N₂O₂S (265.12)]:

Calculated: C 36.24%; H 2.28%; Cl 26.74%; N 10.57%; S 12.09%

Measured: C 36.24%; H 2.36%; Cl 27.38%; N 10.59%; S 11.86%.

### Example 4

### 7,8-Dichloro-4-ethylbenzo[1,2,3]thiadiazine-1,1-dioxide

The title compound is produced according to the procedure of Example 1 starting from 2,3-dichloro-6-propionylbenzenesulfonyl-chloride (26.54 g; 0.088 mol) and hydrazine monohydrate (6.3 ml; 6.5 g; 0.13 mol).

Yield, 21.5 g, white crystals (88%).

Melting point, 238-239° C. (2-propanol)

IR (KBr): 3113 (NH); 1332, 1192 (S═O) cm⁻¹.

¹HNMR (DMSO, 400 MHz): 12.43 (1H, s); 8.14 (1H, d, J=8.8 Hz); 7.93 (1H, d, J=8.8 Hz); 2.93 (2H, q, J=7.4 Hz); 1.17 (3H, t, J=7.4 Hz) ppm.

¹³CNMR (DMSO, 400 MHz): 152.4; 136.2; 134.1; 133.8; 128.0; 127.5; 125.4; 26.3; 11.4 ppm.

Elemental analysis [calculated on the basis of the Formula C₉H₈Cl₂N₂O₂S (279.15)]:

Calculated: C 38.73%; H 2.89%; Cl 25.40%; N 10.04%; S 11.49%

Measured: C 38.81%; H 2.86%; Cl 25.27%; N 10.06%; S 11.35%.

### Example 5

### 7,8-Dichloro-4,5-dimethylbenzo[1,2,3]thiadiazine-1,1-dioxide

The title compound is prepared according to the procedure of Example 1 starting from 2-acetyl-5,6-dichloro-3-methylbenzenesulfonyl-chloride (6.03 g; 0.02 mol) and hydrazine monohydrate (6.6 ml; 6.55 g; 0.131 mol).

Yield, 2.07 g, white crystals (37%).

Melting point, 222-227° C. (ethanol).

IR (KBr): 3224 (NH); 1356, 1157 (S═O) cm⁻¹.

¹HNMR (DMSO, 400 MHz): 12.1 (1H, s); 8.02 (1H, s); 2.62 (3H, s); 2.53 (3H, s) ppm.

¹³CNMR (DMSO, 400 MHz): 152.1; 138.6; 137.1; 135.5; 135.2; 128.1; 123.0; 24.4; 22.7 ppm.

Elemental analysis [calculated on the basis of the Formula C₉H₃Cl₂N₂O₂S (279.15)]:

Calculated: C 38.73%; H 2.89%; Cl 25.40%; N 10.04%; S 11.49%

Measured: C 38.68%; H 2.88%; Cl 25.19%; N 10.03%; S 11.39%.

### Example 6

### 8-chloro-4-methylbenzo[1,2,3]thiadiazine-1,1-dioxide

The title compound is produced according to the procedure of Example 1 starting from 2-acetyl-6-chlorobenzenesulfonylchloride (19.0 g; 0.075 mol) and hydrazine monohydrate (7.3 ml; 7.51 g; 0.15 mol).

Yield, 14.7 g, white crystals (85%).

Melting point, 206-207° C. (2-propanol)

IR (KBr): 3213 (NH); 1319, 1167 (S═O) cm⁻¹.

¹HNMR (DMSO, 200 MHz): 12.23 (1H, s); 7.98-7.83 (4H, m); 2.48 (3H, s) ppm.

¹³CNMR (DMSO, 200 MHz): 148.8; 134.3; 133.8; 131.8; 130.1; 126.9; 126.7; 20.4 ppm.

Elemental analysis [calculated on the basis of the Formula C₈H₇ClN₂O₂S (230.67)]:

Calculated: C 41.66%; H 3.06%; Cl 15.37%; N 12.14%; S 13.90%.

Measured: C 41.75%; H 3.09%; Cl 15.18%; N 12.06%; S 13.80%.

### Example 7

### 8-chloro-4-methyl-7-methoxybenzo[1,2,3]thiadiazine-1,1-dioxide

The title compound is produced according to the procedure described in Example 1 by reacting 6-acetyl-2-chloro-3-methoxybenzene-sulfonylchloride (2.83 g; 0.01 mol) and hydrazine monohydrate (3.4 ml; 3.5 g; 0.07 mol).

Yield, 2.17 g, white crystals (83%).

Melting point, 227-229° C. (acetonitrile)

IR (KBr): 3159 (NH); 1338, 1196 (S═O) cm⁻¹.

¹HNMR (DMSO, 400 MHz): 11.94 (1H, s); 7.84 (1H, d, J=9.0 Hz); 7.60 (1H, d, J=9.0 Hz); 4.02 (3H, s); 2.43 (3H, s) ppm.

¹³CNMR (DMSO, 400 MHz): 157.3; 149.0; 133.5; 128.5; 122.0; 116.2; 114.8; 57.4; 20.3 ppm.

Elemental analysis [calculated on the basis of the Formula C₉H₉ClN₂O₃S (260.70)]:

Calculated: C 41.47%; H 3.48%; Cl 13.60%; N 10.75%; S 12.30%

Measured: C 41.34%; H 3.45%; Cl 13.52%; N 10.68%; S 12.12%.

### Example 8

### 4-Methyl-8-methoxybenzo[1,2,3]thiadiazine-1,1-dioxide

The title compound is produced according to the procedure of Example 1 by starting from 2-acetyl-6-methoxybenzene-sulfonylchloride (5.00 g; 0.02 mol) and hydrazine monohydrate (6.3 ml; 6.50 g; 0.13 mol).

Yield, 3.28 g, white crystals (73%)

Melting point, 194-197° C. (methanol).

IR (KBr): 3205 (NH); 1314, 1169 (S═O) cm⁻¹.

¹HNMR (DMSO, 400 MHz): 11.8 (1H, s); 7.79 (1H, t, J=8.2 Hz); 7.48 (1H, d, J=8.3 Hz); 7.37 (1H, d, J=7.5 Hz); 3.96 (3H, s); 2.43 (3H, s) ppm.

¹³CNMR (DMSO, 400 MHz): 154.2; 147.8; 133.9; 129.3; 122.4; 118.6; 115.5; 56.7; 20.2 ppm.

Elemental analysis [calculated on the basis of the Formula C₉H₁₀N₂O₃S (226.26)]:

Calculated: C 47.78%; H 4.46%; N 12.38%; S 14.17%

Measured: C 47.81%; H 4.44%; N 12.33%; S 13.89%

### Example 9

### 7,8-Dimethoxy-4-methylbenzo[1,2,3]thiadiazine-1,1-dioxide

The title compound is produced according to the procedure of Example 1 starting from 6-acetyl-2,3-dimethoxybenzene-sulfonylchloride (18.1 g; 0.065 mol) and hydrazine monohydrate (6.3 ml; 6.5 g; 0.13 mol).

Yield, 10.2 g, white crystals (61%).

Melting point, 238-240° C. (methanol)

IR (KBr): 3222 (NH); 1326, 1318, 1131(S═O) cm⁻¹.

¹HNMR (DMSO, 500 MHz): 11.57 (1H, s); 7.58 (1H, d, J=8.8 Hz); 7.51 (1H, d, J=8.9 Hz); 3.97 (3H, s); 3.87 (3H, s); 2.39 (3H, s) ppm.

Elemental analysis [calculated on the basis of the Formula C₁₀H₁₂N₂O₄S (256.28)]:

Calculated: C 46.87%; H 4.72%; N 10.93%; S 12.51%.

Measured: C 46.87%; H 4.53%; N 11.08%; S 12.64%.

### Example 10

### 7,8-Dichloro-4,6-dimethylbenzo[1,2,3]thiadiazine-1,1-dioxide

The title compound is produced according to the procedure of Example 1 starting from 2-acetyl-5,6-dichloro-4-methylbenzene-sulfonylchloride (6.03 g; 0.02 mol) and hydrazine monohydrate (9.7 ml; 10.0 g; 0.2 mol).

Yield, 4.25 g, white crystals (76%)

Melting point, 224-226° C. (ethanol).

IR (KBr): 3235 (NH); 1328, 1147 (S═O) cm⁻¹.

¹HNMR (DMSO, 500 MHz): 12.31 (1H, s); 7.89 (1H, d, J=0.7 Hz); 2.55 (3H, d, J=0.6 Hz); 2.47 (3H, s) ppm.

¹³CNMR (DMSO, 500 MHz): 148.5; 142.9; 136.5; 131.3; 128.5; 127.5; 125.4; 21.4; 20.3 ppm.

Elemental analysis [calculated on the basis of the Formula C₉H₈Cl₂N₂O₂S (279.15)]:

Calculated: C 38.73%; H 2.89%; Cl 25.40%; N 10.04%; S 11.49%

Measured: C 38.94%; H 3.01%; Cl 25.48%; N 9.81%; S 11.28%.

### Example 11

### 7,8-Dichlorobenzo[1,2,3]thiadiazine-1,1-dioxide

Procedure “a”

2,3-Dichloro-6-[1,3]dioxolane-2-yl-benzenesulfonylchloride (60.34 g; 0.19 mol) is dissolved in 2-propanol (290 ml) and to this solution acetylhydrazine (28.15 g; 0.38 mol) is added at the temperature of 10° C. After one hour stirring, the crystals are filtered (66.8 g). N-acetyl-N′-(2,3-dichloro-6-[1,3]dioxolane-2-yl-benzenesulfonyl)-hydrazine thus obtained is boiled with aqueous hydrochloric acid solution (10%; 400 ml) for 2.5 hours and filtered.

Yield, 41.52 g, white crystals (87%).

Melting point, 183-185° C. (2-propanol)

IR (KBr): 3242 (NH); 1361, 1175 (S═O) cm⁻¹.

¹HNMR (DMSO, 400 MHz): 8.46 (1H, bs); 7.94 (1H, s); 7.83 (1H, d, J=8.4 Hz); 7.45 (1H, d, J=8.4 Hz) ppm.

Elemental analysis [calculated on the basis of the Formula C₇H₄Cl₂N₂O₂S (251.09)]:

Calculated: C 33.48%; H 1.61%; Cl 28.24%; N 11.16%; S 12.77%.

Measured: C 33.89%; H 1.64%; Cl 27.98%; N 11.06%; S 12.63%.

Procedure “b”

The title compound is prepared according to the procedure of Example 1 starting from 2-formyl-5,6-dichlorobenzenesulfonylchloride (22.12 g; 0.081 mol) and hydrazine monohydrate (7.8 ml; 8.0 g; 0.16 mol).

Yield, 14.0 g, white crystals (69%).

Melting point, 183-185° C. (ethanol)

### Example 12

### 6-Methoxybenzo[1,2,3]thiadiazine-1,1-dioxide

The title compound is produced according to the procedure of Example 1 from 2-formyl-4-methoxybenzenesulfonylchloride (4.69 g; 0.02 mol) and hydrazine monohydrate (9.7 ml; 10.0 g; 0.2 mol).

Yield, 2.37 g, white crystals (56%).

Melting point, 163-164° C. (ethanol).

IR (KBr): 3215 (NH); 1311, 1141 (S═O) cm⁻¹.

¹HNMR (DMSO, 400 MHz): 12.2 (1H, s); 8.21 (1H, s); 7.89 (1H, d, J=8.6 Hz); 7.41 (1H, d, J=2.6 Hz); 7.38 (1H, dd, J=2.6; 8.7 Hz); 3.90 (3H, s) ppm.

¹³CNMR (DMSO, 400 MHz): 162.3; 141.6; 129.6; 126.0; 122.3; 119.3; 111.6; 56.2 ppm.

Elemental analysis [calculated on the basis of the Formula C₈H₈N₂O₃S (212.23)]:

Calculated: C 45.28%; H 3.80%; N 13.20%; S 15.11%

Measured: C 45.36%; H 3.84%; N 12.94%; S 14.96%.

### Example 13

### 4-Methylbenzo[1,2,3]thiadiazine-1,1-dioxide

8-chloro-4-methylbenzo[1,2,3]thiadiazine-1,1-dioxide (2.3 g; 0.01 mol) is dissolved in tetrahydrofurane (20 ml) and the solution is cooled to −78° C. At this temperature, the solution of butyl lithium in hexane (1.6 M solution in hexane; 16 ml; 0.04 mol) is added dropwise. The reaction mixture is stirred at −78° C. for one hour, and thereafter it is poured onto the mixture of ice and water (100 g). The mixture is washed with ethylacetate (60 ml); the aqueous layer is made acidic using concentrated hydrochloric acid, extracted with diethylether (3×50 ml), dried and evaporated.

Yield, 1.8 g, white crystals (92%)

Melting point, 183-185° C. (2-propanol)

IR (KBr): 3229 (NH); 1314, 1173 (S═o) cm⁻¹.

¹HNMR (DMSO, 400 MHz): 12.06 (1H, s); 7.99-7.85 (4H, m); 2.49 (3H, s) ppm.

¹³CNMR (DMSO, 400 MHz): 148.6; 133.6; 133.1; 132.6; 127.7; 127.1; 120.1; 19.8 ppm.

Elemental analysis [calculated on the basis of the Formula C₈H₈N₂O₂S (196.23)]:

Calculated: C 48.97%; H 4.11%; N 14.28%; S 16.34%

Measured: C 49.20%; H 4.20%; N 14.25%; S 16.23%.

### Example 14

### 4-Methyl-7-methoxybenzo[1,2,3]thiadiazine-1,1-dioxide

The title compound is produced according to the procedure described in Example 13 starting from 8-chloro-4-methyl-7-methoxybenzo[1,2,3]thiadiazine-1,1-dioxide (4.16 g; 0.016 mol) and butyl lithium (2.5 M solution in hexane; 20 ml; 0.05 mol).

Yield, 2.27 g, white crystals (63%).

Melting point, 174-176° C. (methanol)

IR (KBr): 3237 (NH); 1314, 1163 (S═O) cm⁻¹.

¹HNMR (DMSO, 400 MHz): 11.85 (1H, s); 7.83 (1H, d, J=8.4 Hz); 7.43-7.38 (2H, m); 3.94 (3H, s); 2.43 (3H, s) ppm.

¹³CNMR (DMSO, 400 MHz): 161.8; 148.6; 135.4; 129.5; 120.9; 119.7; 103.6; 56.4; 19.8 ppm.

Elemental analysis [calculated on the basis of the Formula C₉H₁₀N₂O₃S (226.26)]:

Calculated: C 47.78%; H 4.46%; N 12.38%; S 14.17%

Measured: C 48.03%; H 4.51%; N 12.26%; S 14.08%.

### Example 15

### 7-chlorobenzo[1,2,3]thiadiazine-1,1-dioxide

The title compound is produced according to the process of Example 14 starting from 7,8-dichlorobenzo[1,2,3]thiadiazin-1,1-dioxide (4.01 g; 0.016 mol) and butyl lithium (2.5 M solution in hexane, 20 ml; 0.05 mol).

Yield, 1.31 g, white crystals (38%).

Melting point, 221-223° C. (ethanol).

IR (KBr): 3230 (NH); 1331, 1170 (S═O) cm⁻¹.

¹HNMR (DMSO, 400 MHz): 12.63 (1H, s); 8.35 (1H, d, J=0.5 Hz); 8.08 (1H, dt, J=0.5; 2.1 Hz); 7.99 (1H, dd, J=2.1; 8.4 Hz); 7.91 (1H, d, J=8.3 Hz) ppm.

¹³CNMR (DMSO, 400 MHz): 141.7; 136.9; 134.3; 133.4; 130.4; 126.3; 119.9 ppm.

Elemental analysis [calculated on the basis of the Formula C₇H₅ClN₂O₂S (216.65)]:

Calculated: C 38.81%; H 2.33%; Cl 16.36%; N 12.93%; S 14.80%

Measured: C 39.16%; H 2.28%; Cl 16.29%; N 12.69%; S 14.52%.

### Example 16

### 2,4-Dimethyl-7-chlorobenzo[1,2,3]thiadiazine-1,1-dioxide

7-chloro-4-methylbenzo[1,2,3]thiadiazine-1,1-dioxide (4.61 g; 0.02 mol) is dissolved in N,N-dimethylformamide (20 ml) and to this solution potassium-tert-butylate (4.4 g; 0.04 mol) is added at 10° C. Thereafter methyl iodide (2.5 ml; 5.6 g; 0.04 mol) is added dropwise to the reaction mixture. The mixture is stirred for one hour at 50° C. and poured onto water-ice mixture. The crystals thus formed are filtered and washed with water.

Yield, 3.57 g, white crystals (75%).

Op.: 116-117° C.

IR (KBr): 1590 (C═N); 1328, 1178 (S═O) cm⁻¹.

¹HNMR (CDCl₃, 400 MHz): 7.97 (1H, d, J=2.1 Hz); 7.70 (1H, dd, J=2.1; 8.5 Hz); 7.58 (1H, d, J=8.5 Hz); 3.53 (3H, s); 2.50 (3H, s) ppm.

Elemental analysis [calculated on the basis of the Formula C₉H₉ClN₂O₂S (244.70)]:

Calculated: C 44.18%; H 3.71%; Cl 14.49%; N 11.45%; S 13.10%

Measured: C 44.02%; H 3.70%; Cl 14.43%; N 11.48%; S 13.02%.

### Example 17

### 7,8-Dichloro-2,4-dimethylbenzo[1,2,3]thiadiazine-1,1-dioxide

The title compound is prepared according to the procedure of Example 16 using 7,8-dichloro-4-methylbenzo[1,2,3]thiadiazine-1,1-dioxide (2.65 g; 0.01 mol); potassium-tent-butylate (2.24 g 0.02 mol) and methyl iodide (1.3 ml; 2.8 g; 0.02 mol).

Yield, 2.5 g, white crystals (90%)

Melting point, 220-222° C. (2-propanol)

IR (KBr): 1334, 1158 (S═O) cm⁻¹.

¹HNMR (CDCl₃, 400 MHz): 7.79 (1H, d, J=8.6 Hz); 7.49 (1H, d, J=8.6 Hz); 3.55 (3H, s); 2.49 (3H, s) ppm.

¹³CNMR (CDCl₃, 400 MHz): 147.2; 137.6; 136.5; 132.9; 128.9; 127.8; 125.5; 39.2; 20.6 ppm.

Elemental analysis [calculated on the basis of the Formula C₉H₈Cl₂N₂O₂S (279.15)]:

Calculated: C 38.73%; H 2.89%; Cl 25.40%; N 10.04%; S 11.49%

Measured: C 38.90%; H 3.05%; Cl 25.08%; N 10.02%; S 11.51%.

### Example 18

### 7,8-Dichloro-4-ethyl-2-methylbenzo[1,2,3]thiadiazine-1,1-dioxide

The title compound is produced according to the procedure of Example 16 by starting from 7,8-dichloro-4-ethylbenzo[1,2,3]thiadiazine-1,1-dioxide (11.2 g; 0.04 mol); potassium-tent-butylate (9.0 g 0.08 mol) and methyl iodide (5.0 ml; 11.4 g; 0.08 mol).

Yield, 10.2 g, white crystals (87%).

Melting point, 119-120° C. (methanol).

IR (KBr): 1329, 1158 (S=0) cm⁻¹.

¹HNMR (CDCl₃, 500 MHz): 7.79 (1H, d, J=8.6 Hz); 7.52 (1H, d, J=8.6 Hz); 3.56 (3H, s); 2.86 (2H, q, J=7.4 Hz); 1.27 (3H, t, J=7.4 Hz) ppm.

¹³CNMR (CDCl₃, 125.6 MHz): 151.5; 137.7; 133.7; 133.5; 128.6; 128.2; 125.3; 36.5; 27.3; 11.8 ppm.

Elemental analysis [calculated on the basis of the Formula C₁₀H₁₀Cl₂N₂O₂S (293.17)]:

Calculated: C 40.97%; H 3.44%; Cl 24.19%; N 9.56%; S 10.94%

Measured: C 41.24%; H 3.47%; Cl 23.84%; N 9.54%; S 10.90%.

### Example 19

### 2,4-Diethyl-7,8-dichlorobenzo[1,2,3]thiadiazine-1,1-dioxide

The title compound is prepared according to the procedure of Example 16 using 7,8-dichloro-4-ethylbenzo[1,2,3]thiadiazine-1,1-dioxide (1.0 g; 0.0036 mol); potassium-tert-butylate (0.8 g 0.0072 mol) and ethyl iodide (0.56 ml; 1.1 g; 0.0072 mol).

Yield, 0.75 g, white crystals (68%).

Melting point, 79-82° C. (methanol).

IR (KBr): 1334, 1152 (S=0) cm⁻¹.

¹HNMR (CDCl₃, 200 MHz): 7.78 (1H, d, J=8.6 Hz); 7.52 (1H, d, J=8.6 Hz); 3.97 (2H, q, J=7.1 Hz); 2.88 (2H, q, J=7.3 Hz); 1.43 (3H, t, J=7.1 Hz); 1.28 (3H, t, J=7.3 Hz) ppm.

Elemental analysis [calculated on the basis of the Formula C₁₁H₁₂Cl₂N₂O₂S (307.20)]:

Calculated: C 43.01%; H 3.94%; Cl 23.08%; N 9.12%; S 10.44%

Measured: C 43.12%; H 3.97%; Cl 22.97%; N 9.11%; S 10.35%.

### Example 20

### 7,8-Dichloro-2-methylbenzo[1,2,3]thiadiazine-1,1-dioxide

Potassium-tert-butylate (1.12 g; 0.01 mol) is suspended in N,N-dimethylformamide (5 ml) and to this suspension the solution of 7,8-dichlorobenzo[1,2,3]thiadiazine-1,1-dioxide (1.26 g; 0.005 mol) in N,N-dimethylformamide (15 ml) is added at room temperature. The suspension is stirred for 30 minutes, thereafter methyl iodide (2.13 g; 0.93 ml; 0.015 mol) is added dropwise. The mixture is stirred for an hour, subsequently poured into water and the crystals are filtered. The crystalline mass thus obtained is stirred with diethylether (25 ml) for one hour, filtered, the filtrate containing 7,8-dichloro-2-methylbenzo[1,2,3]thiadiazine-1,1-dioxide is evaporated. The residue is filtered from ethanol.

Yield, 0.67 g, yellow crystals (50%)

Melting point, 120-121° C. (hexane-ethylacetate 1:1).

IR (KBr): 1338, 1178 (S═O) cm⁻¹.

¹HNMR (DMSO, 400 MHz): 8.37 (1H, s); 8.20 (1H, d, J=8.4 Hz); 7.89 (1H, d, J=8.4 Hz); 3.53 (3H, s) ppm.

¹³CNMR (DMSO, 400 MHz): 141.3; 136.7; 135.0; 131.4; 129.1; 128.3; 125.4; 36.1 ppm.

Elemental analysis [calculated on the basis of the Formula C₈H₆Cl₂N₂O₂S (265.12)]:

Calculated: C 36.24%; H 2.28%; Cl 26.74%; N 10.57%; S 12.09%

Measured: C 36.35%; H 2.38%; Cl 27.04%; N 10.65%; S 11.84%.

### Example 21

### 7-chloro-2-methylbenzo[1,2,3]thiadiazine-1,1-dioxide

Sodium hydride (2.63 g; 0.055 mol) is suspended in tetrahydrofurane (50 ml) and into this suspension is added dropwise the solution of 7-chlorobenzo[1,2,3]thiadiazine-1,1-dioxide (10.83 g; 0.05 mol) in tetrahydrofurane (150 ml). The suspension is stirred for 30 minutes, thereafter methyl iodide is added dropwise (9.3 ml; 21.30 g; 0.150 mol). The reaction mixture is stirred for one hour, acetone is added and the thus obtained mixture is stirred further until dissolution. The mixture is evaporated, the residue is mixed with water and filtered. The raw product is stirred with tetrahydrofurane at room temperature, filtered and the filtrate is evaporated.

Yield, 3.9 g, yellow crystals (34%).

Melting point, 131-133° C. (hexane-ethylacetate 1:1).

IR (KBr): 1321, 1172 (S═O) cm⁻¹.

¹HNMR (DMSO, 200 MHz): 8.41 (1H, s); 8.17 (1H, d, J=1.5 Hz); 8.03 (1H, dd, J=1.8; 8.6 Hz); 7.94 (1H, d, J=8.3 Hz); 3.50 (3H, s) ppm.

Elemental analysis [calculated on the basis of the Formula C₈H₇ClN₂O₂S (230.67)]:

Calculated: C 41.66%; H 3.06%; Cl 15.37%; N 12.14%; S 13.90%

Measured: C 41.04%; H 3.09%; Cl 15.01%; N 12.01%; S 13.72%.

### Example 22

### 2-Methyl-6-methoxybenzo[1,2,3]thiadiazine-1,1-dioxide

The title compound is prepared according to the procedure described in Example 16 starting from 6-methoxybenzo[1,2,3]thiadiazine-1,1-dioxide (2.12 g; 0.01 mol), potassium-tert-butylate (2.24 g 0.02 mol) and methyl iodide (1.2 ml; 2.8 g; 0.02 mol).

Yield, 0.63 g, yellow crystals (28%).

Melting point, 99-101° C. (hexane-ethylacetate 1:1).

IR (KBr): 1313, 1167 (S═O) cm⁻¹.

¹HNMR (CDCl₃, 400 MHz): 7.91 (1H, d, J=9.1 Hz); 7.89 (1H, s); 7.22 (1H, dd, J=2.58.7 Hz); 6.96 (1H, d, J=2.5 Hz); 3.98 (3H, s); 3.58 (3H, s) ppm.

¹³CNMR (CDCl₃, 400 MHz): 162.8; 140.1; 130.0; 125.1; 123.4; 118.7; 110.5; 55.9; 35.3 ppm.

Elemental analysis [calculated on the basis of the Formula C₉H₁₀N₂O₃S (226.26)]:

Calculated: C 47.78%; H 4.46%; N 12.38%; S 14.17%

Measured: C 47.37%; H 4.50%; N 12.03%; S 14.01%.

### Example 23

### 2-Methylbenzo[1,2,3]thiadiazine-1,1-dioxide

The title compound is prepared according to the procedure of Example 16 starting from benzo[1,2,3]thiadiazin-1,1-dioxide (1.82 g; 0.01 mol; for preparation, see: King, J. F.; Huston, B. L.; Hawson, A., Deaken, D. M., Harding, D. R. K. Can. J. Chem., 1971, 49, 943.), potassium-tert-butylate (2.5 g 0.02 mol) and methyl iodide (1.3 ml; 2.8 g; 0.02 mol) with the modification that the product is isolated by pouring the reaction mixture into water, extracting with diethylether, evaporating the extract and filtering the residue from hexane.

Yield, 0.63 g, white crystals (32%).

Melting point, 79-80° C. (hexane-ethylacetate 1:1).

IR (KBr): 13320175 (S═O) cm⁻¹.

¹HNMR (CDCl₃, 200 MHz): 8.03-7.96 (2H, m); 7.81-7.70 (2H, m); 7.61-7.52 (1H, m); 3.61 (3H, s) ppm.

¹³CNMR (CDCl₃, 200 MHz): 140.7; 132.7; 132.4; 132.0; 128.0; 127.0; 121.1; 35.5 ppm.

Elemental analysis [calculated on the basis of the Formula C₈H₈N₂O₂S (196.23)]:

Calculated: C 48.97%; H 4.11%; N 14.28%; S 16.34%

Measured: C 48.79%; H 4.14%; N 14.22%; S 16.11%.

### Example 24

### 7,8-Dichloro-2,4,5-trimethylbenzo[1,2,3]thiadiazine-1,1-dioxide

The title compound is produced by the procedure of Example 16 starting from 7,8-dichloro-4,5-dimethylbenzo [1,2,3]thiadiazine-1,1-dioxide (2.79 g; 0.01 mol), potassium-tert-butylate (2.24 g 0.02 mol) and methyl iodide (1.9 ml; 4.25 g; 0.03 mol).

Yield, 5.17 g, white crystals (88%)

Melting point, 166-168° C. (ethanol).

IR (KBr): 1327, 1149 (S═O) cm⁻¹.

¹HNMR (CDCl₃, 200 MHz): 7.60 (1H, s); 3.47 (3H, s); 2.58 (3H, s); 2.55 (3H, s) ppm.

Elemental analysis [calculated on the basis of the Formula C₁₀H₁₀Cl₂N₂O₂S (293.19)]:

Calculated: C 40.97%; H 3.44%; Cl 24.18%; N 9.56%; S 10.94%

Measured: C 41.28%; H 3.46%; Cl 24.26%; N 9.28%; S 10.75%.

### Example 25

### 8-chloro-7-methoxy-2,4-dimethylbenzo[1,2,3]thiadiazine-1,1-dioxide

The title compound is produced according to the procedure of Example 16 using 8-chloro-4-methyl-7-methoxybenzo[1,2,3]thiadiazine-1,1-dioxide (3.91 g; 0.015 mol), potassium-tert-butylate (3.37 g; 0.03 mol) and methyl iodide (2.8 ml; 6.4 g; 0.045 mol).

Yield, 3.74 g, white crystals (91%)

Melting point, 151-152° C. (ethanol).

IR (KBr): 1323, 1194 (S═O) cm⁻¹.

¹HNMR (CDCl₃, 400 MHz): 7.53 (1H, d, J=8.9 Hz); 7.21 (1H, d, J=8.9 Hz); 4.01 (3H, s); 3.51 (3H, s); 2.45 (3H, s) ppm.

Elemental analysis [calculated on the basis of the Formula C₁₀H₁₁ClN₂O₃S (274.74)]:

Calculated: C 43.72%; H 4.04%; Cl 12.90%; N 10.20%; S 11.67%

Measured: C 43.60%; H 4.10%; Cl 12.98%; N 10.15%; S 11.58%.

### Example 26

### 7,8-Dichloro-2,4,6-trimethylbenzo[1,2,3]thiadiazine-1,1-dioxide

The title compound is prepared according to the procedure of Example 16 starting from 7,8-dichloro-4,6-dimethylbenzo[1,2,3]thiadiazine-1,1-dioxide (2.79 g; 0.01 mol), potassium-tert-butylate (2.24 g; 0.02 mol) and methyl iodide (1.9 ml; 4.25 g; 0.03 mol).

Yield, 1.75 g, white crystals (95%)

Melting point, 179-180° C. (ethanol).

IR (KBr): 1355, 1183 (S═O) cm⁻¹. ¹HNMR (CDCl₃, 200 MHz): 7.96 (1H, s); 3.42 (3H, s); 2.57 (3H, s); 2.49 (3H, s) ppm.

Elemental analysis [calculated on the basis of the Formula C₁₀H₁₀Cl₂N₂O₂S (293.19)]:

Calculated: C 40.97%; H 3.44%; Cl 24.18%; N 9.56%; S 10.94%

Measured: C 40.67%; H 3.44%; Cl 24.06%; N 9.32%; S 11.17%.

### Example 27

### 8-chloro-7-methoxybenzo[1,2,3]thiadiazine-1,1-dioxide

The title compound is produced according to the procedure described in Example 1 starting from 2-formyl-6-chloro-5-methoxybenzenesulfonylchloride (2.69 g; 0.01 mol) and hydrazine monohydrate (2.4 ml; 2.5 g; 0.05 mol).

Yield, 1.66 g, white crystals (67%).

Op.: 189-191° C. (ethyl acetate).

IR (KBr): 3292 (NH); 1282, 1160 (S═O) cm⁻¹.

¹HNMR (DMSO, 400 MHz): 12.29 (1H, s); 8.19 (1H, s); 7.84 (1H, d, J=8.8 Hz); 7.66 (1H, d, J=8.8 Hz); 4.02 (3H, s) ppm.

Elemental analysis [calculated on the basis of the Formula C₈H₇ClN₂O₃S (246.67)]:

Calculated: C, 38.95; H, 2.86; Cl 14.37; N 11.36; S 13.00%

Measured: C, 39.23; H, 2.80; Cl 14.38; N 10.97; S 13.04%.

### Example 28

### 8-chloro-2-methyl-7-methoxybenzo[1,2,3]thiadiazine-1,1-dioxide

The title compound is prepared according to the procedure of Example 16 using 8-chloro-7-methoxy-benzo[1,2,3]thiadiazine-1,1-dioxide (1.23 g; 0.005 mol); potassium-tent-butylate (1.12 g; 0.01 mol) and methyl iodide (0.65 ml; 1.4 g; 0.01 mol).

Yield, 0.97 g, white crystals (74%).

Melting point, 173-175° C. (ethanol)

IR (KBr): 1333, 1174 (S═O) cm⁻¹.

¹HNMR (DMSO, 400 MHz): 8.24 (1H, s); 7.86 (1H, d, J=8.8 Hz); 7.70 (1H, d, J=8.8 Hz); 4.02 (3H, s) 3.45 (3H, s) ppm.

Elemental analysis [calculated on the basis of the Formula C₉H₉ClN₂O₃S (260.70)]:

Calculated: C, 41.47; H, 3.48; Cl 13.60; N 10.75; S 12.30%

Measured: C, 41.40; H, 3.40; Cl 13.45; N 10.65; S 12.34%.

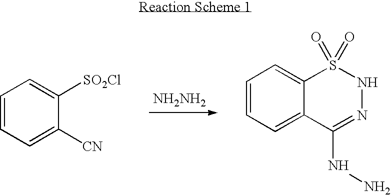

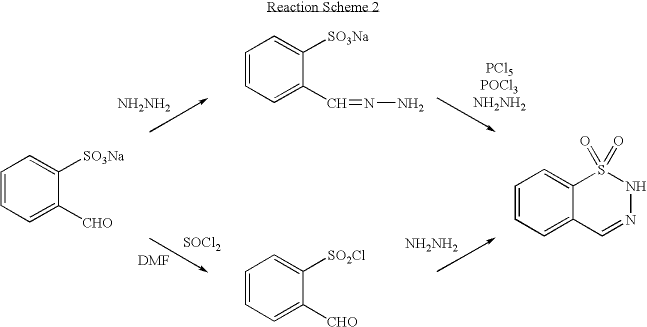

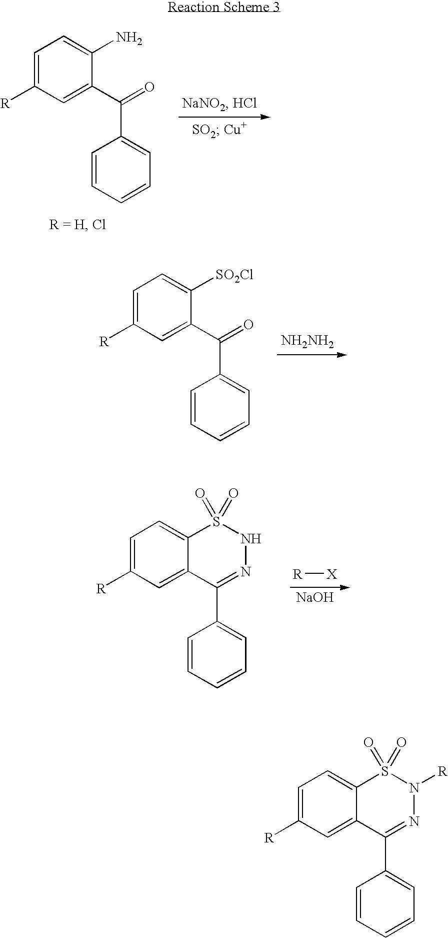

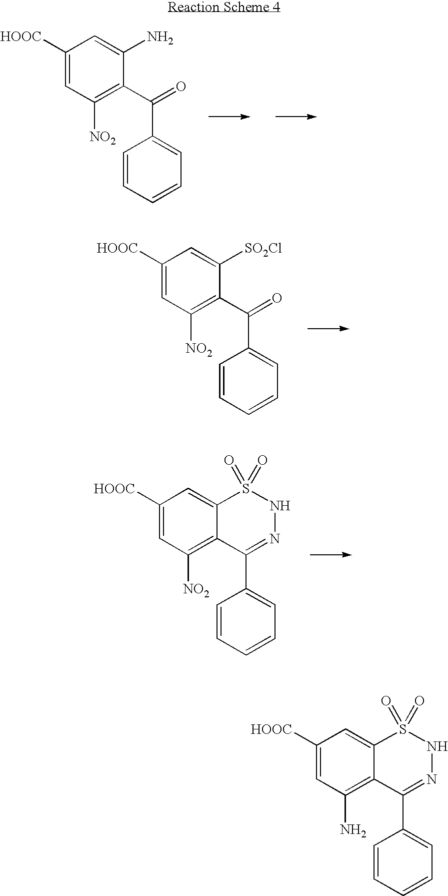

1. Benzo[1,2,3]thiadiazine-1,1-dioxide derivatives of the Formula (I), 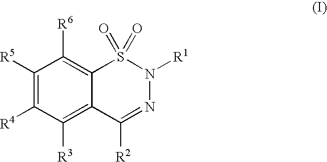
wherein
R1 and R2 each represents hydrogen or a straight- or branched-chain alkyl group containing 1 to 4 carbon atoms
R3, R4, R5 and R6 each represents hydrogen, halogen, a straight- or branched-chain alkyl group having 1 to 4 carbon atoms or an alkoxy group containing a straight- or branched-chain alkyl having 1 to 4 carbon atoms.

2. 7,8-Dichloro-4-methylbenzo[1,2,3]thiadiazine-1,1-dioxide as defined in claim 1.

3. 7,8-Dichloro-4,5-dimethylbenzo[1,2,3]thiadiazine-1,1-dioxide as defined in claim 1.

4. 2,4-Dimethyl-7-chlorobenzo[1,2,3]thiadiazine-1,1-dioxide as defined in claim 1.

5. 2,4-Diethyl-7,8-dichlorobenzo[1,2,3]thiadiazine-1,1-dioxide as defined in claim 1.

6. Process for the preparation of berizo[1,2,3]thiadiazine-1,1-dioxide derivatives, of the Formula (I) as defined in claim 1 wherein R1 and R2 each represents hydrogen or a straight- or branched-chain alkyl having 1 to 4 carbon atoms; R3, R4, R5 and R6 represent hydrogen, halogen, a straight- or branched-chain alkyl group having 1 to 4 carbon atoms or an alkoxy group containing a branched- or straight chain alkyl having 1 to 4 carbon atoms, which comprises
a) for the preparation of the compounds of the Formula (I) wherein R1 is hydrogen, R2, R3, R4, R5 and R6 is defined hi claim 1, reacting a 2-acyl benzenesulfonylchloride derivative of the Formula (II) 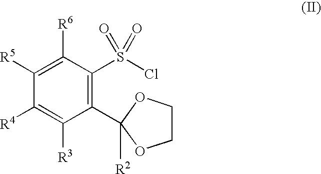
wherein the meaning of R2, R3, R4, R5 and R6 is the above, with a hydrazine derivative, preferably with acetyl-hydrazine and reacting the thus obtained product with a strong acid, preferably with hydrochloric acid; or
b) for the preparation of the compounds of the Formula (I), wherein R1 is hydrogen, the meaning of R2, R3, R4, R5 and R6 is as defined in claim 1, reacting a 2-acyl-benzenesulfonylchloride derivative of the Formula (III) 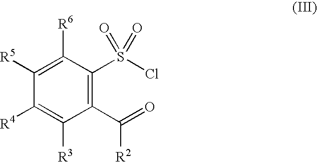
wherein the meaning of R2, R3, R4, R5 and R6 is as defined above, with a hydrazine derivative; or
c) for the preparation of the compounds of the Formula (I), wherein R1 and R6 represent hydrogen and the meaning of R2, R3, R4, R5 is as defined in claim 1, reacting a benzo[1,2,3]thiadiazine-1,1-dioxide derivative wherein R1 represents hydrogen, R6 is halogen, preferably chloride and the meaning of R2, R3, R4, R5 is the above, with alkyl lithium and converting the thus obtained metal-organic compound by reacting said compound with water, or
d) for the preparation of the benzo[1,2,3]thiadiazine-1,1-dioxide derivatives of the Formula (I), wherein R1 represents a straight- or branched-chain alkyl group having 1 to 4 carbon atoms, the meaning of R2, R3, R4, R5, R6 is as defined in claim 1, reacting a benzo[1,2,3]thiadiazine-1,1-dioxide derivative of the Formula (I) wherein R1 represents hydrogen, the meaning of R2, R3, R4, R5 and R6 is as defined above with an alkyl halogenide.

7. Process variant d) of claim 6, which comprises using an alkyl ? chloride, alkyl bromide or alkyl iodide as alkyl halogenide.

8. (canceled)

9. A medicament suitable for the prevention or treatment of anxiety, which comprises a benzo[1,2,3) thiadiazine-1,1-dioxide derivative of the Formula (I) as defined in claim 1 wherein R1 and R2 each represents hydrogen, a straight- or branched-chain alkyl group having 1 to 4 carbon atoms; the meaning of R3, R4, R5 and R6 is hydrogen, halogen, a straight- or branched-chain alkyl group having 1 to 4 carbon atoms or an alkoxy group containing a straight- or branched-chain alkyl group having 1 to 4 carbon atoms and one or more pharmaceutically acceptable vehicle(s) or optionally auxiliary agents.

10. A medicament suitable for the treatment or prevention of generalized anxiety disorder, panic disorder, phobias, compulsive disorders including obsessive-compulsive disorder, posttraumatic stress disorder, stress-induced anxiety condition, comprising a benzo[1,2,3]thiadiazine-1,1-dioxide derivative of the Formula (I) according to claim 1 and one or more pharmaceutically acceptable vehicle(s) or auxiliary agent(s).

11. A medicament suitable for the prevention or treatment of attention-deficient hyperactivity disorder, stress-induced adaptation disorder, posttraumatic stress disorder, anorexia nervosa, bulimia nervosa, insomnia, parasomnia, compulsive disorders, disturbances of the sexual function and the symptoms related to the use or withdrawal of chemical agents including alcohol, caffeine, nicotine, drugs of abuse, sedatives, hypnotic and doping agents comprising a benzo[1,2,3]thiadiazine-1,1-dioxide derivative of the Formula (I) according to claim 1 and one or more pharmaceutically acceptable vehicle(s) or auxiliary agen(s)t.

12. (canceled)

13. (canceled)

14. (canceled)

15. Method for the treatment of the diseases, disorders or conditions of the central nervous system, especially of anxiety, generalized anxiety disorder, panic disorder, phobias, compulsive disorders, posttraumatic stress disorder, stress-induced anxiety disorder, attention-deficit hyperactivity disorder, stress-related adaptation disorder, anorexia nervosa, bulimia nervosa, insomnia, parasomnia, disturbances of the sexual function and the symptoms of the use or withdrawal of chemical agents including alcohol, caffeine, nicotine, sedatives, narcotics, drugs of abuse or doping agents, which comprises administering to a patient in need of such treatment a therapeutically effective amount of benzo[1,2,3]thiadiazine-1,1-dioxide derivative of the Formula (I) according to claims 1.
